(19) United States
(12) Patent Application Publication
(10) Pub. No.: US 2018/0344503 A1
(43) Pub. Date: Dec. 6, 2018

(54) POSTURAL ALIGNMENT APPARATUS

(71) Applicant: Sam Akinrinade, New York, NY (US)

(72) Inventor: Sam Akinrinade, New York, NY (US)

(21) Appl. No.: 15/608,045

(22) Filed: May 30, 2017

(51) Int. Cl. A61F 5/02

(52) U.S. Cl. CPC A61F 5/026; A61F 2005/0158; A61F 5/02

(57) ABSTRACT

A postural alignment apparatus is provided. The postural alignment apparatus is adapted to cue proper spinal alignment during exercise for complex exercises and posture-related activities, while freeing up the wearer's hands. The postural alignment apparatus may include a torso-wear component adapted to be worn along the torso of a human wearer, wherein the torso-wear component provides a spinal portion adapted to be located adjacent along a wearer's spine when the torso-wear component is worn. An elongated member is attached to the spinal portion so as to extend the length of the wearer's spine, enabling spinal alignment.

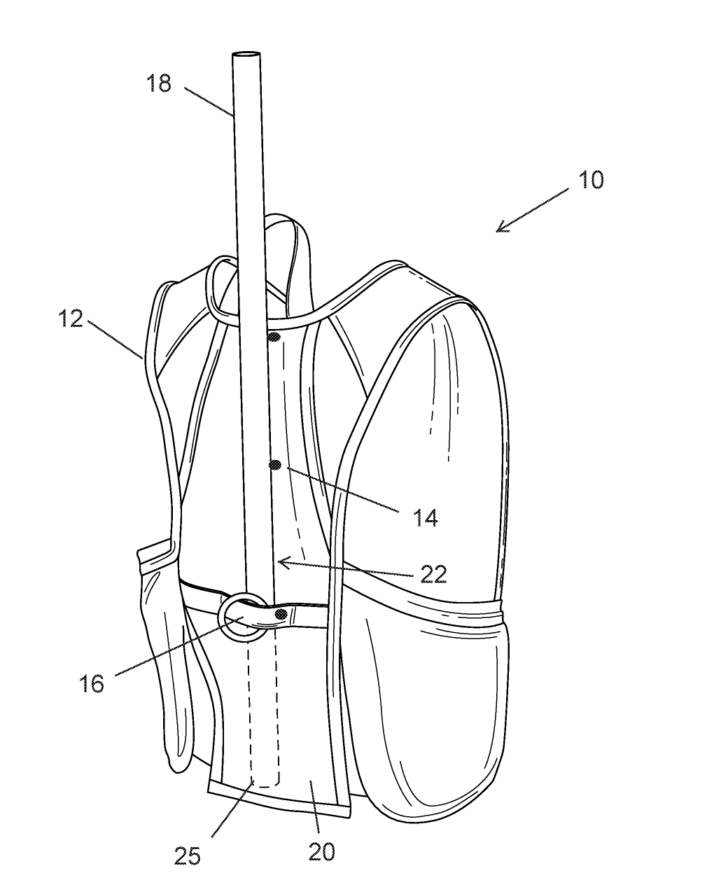

### BACKGROUND OF THE INVENTION

The present invention relates to postural alignment and, more particularly, to a postural alignment apparatus that can be worn while performing exercises.

As a personal trainer/performance coach, the Applicant understands that good posture is key for most exercise movements. Hypothetically, an individual exercising could hold a light stick (such as a dowel or broomstick) along their spine to facilitate good postural awareness while doing exercise movements, but it would limit use of their hand(s) and arm(s) that are occupied holding the stick behind their backs, and thus they could not hold weights or use their arms effectively (e.g., for balance) during more complex movements. Further, the awareness and benefits of the stick for posture would be lost once it is taken away.

Currently, the only other product that tries to address this problem is a belt with some form of back support. The belt concept lacks an elongated member that is secured in place along the spine and does not properly enable the wearer to have full postural awareness, particularly of the mid and upper back. Further, while the wearer does complex movements, a belt consistently requires readjustment of the back support that tends to move, thereby distracting the user during their functional exercises and so frustrating the purpose of freeing up the hands and arms.

As can be seen, there is a need for a postural alignment apparatus that can be worn while preforming exercise routines. The postural alignment apparatus provides lightweight torso-wear similar to ones worn by athletes when they workout. Removably attached at spaced apart attachment points along a portion of the torso-wear adjacent to the spine of a wearer is an elongated member, facilitating and cuing proper spinal alignment during exercise routines, while freeing up the wearer's hands.

### SUMMARY OF THE INVENTION

In one aspect of the present invention, a postural alignment apparatus includes a torso-wear component adapted to be worn along a torso of a human wearer, wherein the torso-wear component provides a spinal portion adapted to be located adjacent a wearer's spine for the length of the spinal portion when the torso-wear component is worn; and at least three attachment points spaced apart along the spinal portion, wherein one of the at least three attachment points is located adjacent the wearer's lumbar spine, and wherein another of the at least three attachment points is located adjacent the wearer's cervical spine.

In another aspect of the present invention, the postural alignment apparatus includes a torso-wear component adapted to be worn along a torso of a human wearer, wherein the torso-wear component provides a spinal portion adapted to be located adjacent a wearer's spine for the length of the spinal portion when the torso-wear component is worn; at least three attachment points spaced apart along the spinal portion, wherein one of the at least three attachment points is located adjacent the wearer's lumbar spine, and wherein another of the at least three attachment points is located adjacent the wearer's cervical spine; a removable fastener provided by each attachment point; and an elongated member extending a length of the wearer's spine, wherein the elongated member has a uniform width of between one to three inches.

Wherein certain embodiments, each removable fastener is a hook and loop fastener; an upward facing pocket is provided wherein the upward facing pocket is adapted to be located adjacent a wearer's waistline, and wherein the at least three attachment points are evenly spaced apart.

These and other features, aspects and advantages of the present invention will become better understood with reference to the following drawings, description and claims.

### BRIEF DESCRIPTION OF THE DRAWINGS

The FIGURE is a perspective view of an exemplary embodiment of the present invention, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a postural alignment apparatus adapted to cue proper spinal alignment during exercise for complex exercises and posture-related activity, while freeing up the wearer's hands. The postural alignment apparatus may include a torso-wear component adapted to be worn along the torso of a human wearer, wherein the torso-wear component provides a spinal portion adapted to be located adjacent along a wearer's spine when the torso-wear component is worn. An elongated member is attached to the spinal portion so as to extend the length of the wearer's spine, enabling spinal alignment.

Referring to the FIGURE, the present invention may include a postural alignment apparatus 10 adapted to cue proper spinal alignment during exercise for complex exercises and posture-related activity, while freeing up the wearer's hands. The postural alignment apparatus 10 may include a torso-wear component 12 adapted to be worn along the torso of a human wearer.

An elongated member 18 may be dimensioned and adapted to extend the length of the wearer's back from approximately their waistline to their neck or even upper neck. The elongated member 18 may generally range from three to four feet in length while having a thickness or diameter of approximately one to three inches. The elongated member 18 may be made of solid or hollow material that can be repeatedly subjected to flexing without warping or failure. In certain embodiments, there would be a plurality of elongated members 18 for a user to choose from, including ones of varying weights so as to be used to increase resistance and exercise difficulty during certain exercise routines.

The torso-wear component 12 may take any shape, e.g., vest, jacket, or the like as long as it can be worn along the torso and provides a plurality of attachment points 14 spaced apart along a spinal portion 22 of the torso-wear component 12, wherein the spinal portion 22 is adapted to be located adjacent along a wearer's spine when said torso-wear component 12 is worn. Each attachment point 14 may provide removable fasteners 16, such as hook and loop fastener loops or the like. The removable fasteners 16 makes for simple and fast attachment or reattachment if a wearer wants to use a different elongated member 18 or raised and lower it along the spinal portion 22. The plurality of attachment points 14 may include at least three removable fasteners 16 spaced along the spinal portion 22 from the lumbar spine to the cervical spine of the wearer. Even though in the FIGURE a removable fastener 16 is shown at only one attachment point 14, it is understood that at each of the plurality of attachment points 14 an associated removable fastener 16 may be disposed. Similarly, even though only three attachment points 14 are shown in the figures, there could be more or less.

The material of the torso-wear component 12 may be lightweight yet durable, similar to what athletes wear when they workout, so as to function herein as described—athletic wear capable of securing the elongated member 18 without failure during an intense exercise routine.

The torso-wear component 12 may also include a pocket 20 for slidable reception of a distal end 25 of the elongated member 18. The pocket 20 may be disposed along an outer surface of the spinal portion 22 adjacent to a waistline of the wearer. In certain embodiments, the pocket 20 would be upward facing relative to the torso-wear component 12 so that the pocket 20 could act as a support of the distal end 25 of the elongated member 18.

A method of using the present invention may include the following. The postural alignment apparatus 10 disclosed above may be provided. A user may secure the desired elongated member 18 in the removable fasteners 16 along the spinal portion 22, including in certain embodiments, by sliding the distal end 25 of the elongated member 18 into the pocket 20. The user would wear the torso-wear component 12, and then proceed to engage in exercises, physical therapy, chiropractic or other rehabilitation activity that addresses posture generally and or spinal alignment specifically. Therefore, the present invention may be advantageous for use in gyms to coach proper alignment during popular movements like squats and deadlifts, or used at home or office to remind the wearer to sit and stand up straight. The present invention is an improvement over the prior art because the spinal support is far more secure as the elongated member 18 extends coextensively from at least the lumbar spine to the cervical spine of the wearer, by being secured by at least three points along the spinal column from bottom of neck to lower back.

It should be understood, of course, that the foregoing relates to exemplary embodiments of the invention and that modifications may be made without departing from the spirit and scope of the invention as set forth in the following claims.

What is claimed is:

1. A postural alignment apparatus, comprising:
a torso-wear component adapted to be worn along a torso of a human wearer, wherein the torso-wear component provides a spinal portion adapted to be located adjacent a wearer's spine for the length of the spinal portion when the torso-wear component is worn; and
at least three attachment points spaced apart along the spinal portion, wherein one of the at least three attachment points is located adjacent the wearer's lumbar spine, and wherein another of the at least three attachment points is located adjacent the wearer's cervical spine.

2. The postural alignment apparatus of claim 1, wherein the at least three attachment points are evenly spaced apart.

3. The postural alignment apparatus of claim 1, further compromising an elongated member extending a length of the wearer's spine.

4. The postural alignment apparatus of claim 1, the elongated member has a uniform width of between one to three inches.

5. The postural alignment apparatus of claim 1, wherein each attachment point provides a removable fastener.

6. The postural alignment apparatus of claim 5, wherein the removable fastener is a hook and loop fastener.

7. The postural alignment apparatus of claim 5, wherein the removable fastener is a hook and loop fastener.

8. The postural alignment apparatus of claim 1, further compromising an upward facing pocket, wherein the upward facing pocket is adapted to be located adjacent a wearer's waistline.

9. A postural alignment apparatus, comprising:
a torso-wear component adapted to be worn along a torso of a human wearer, wherein the torso-wear component provides a spinal portion adapted to be located adjacent a wearer's spine for the length of the spinal portion when the torso-wear component is worn;
at least three attachment points spaced apart along the spinal portion, wherein one of the at least three attachment points is located adjacent the wearer's lumbar spine, and wherein another of the at least three attachment points is located adjacent the wearer's cervical spine;
a removable fastener provided by each attachment point; and
an elongated member extending a length of the wearer's spine, wherein the elongated member has a uniform width of between one to three inches.

10. The postural alignment apparatus of claim 9, wherein each removable fastener is a hook and loop fastener.

11. The postural alignment apparatus of claim 9, further compromising an upward facing pocket, wherein the upward facing pocket is adapted to be located adjacent a wearer's waistline.

12. The spinal alignment apparatus of claim 1, wherein the at least three attachment points are evenly spaced apart.
